(19) United States
(12) Patent Application Publication
(10) Pub. No.: US 2025/0361473 A1
(43) Pub. Date: Nov. 27, 2025

(54) CELL CULTURE STRUCTURE AND CELL CULTURE DEVICE

(71) Applicant: Beyond Manufacture Inc., New Taipei City (TW)

(72) Inventors: Chun-Hao Luo, New Taipei City (TW); Todd Juan, New Taipei City (TW); Jye-Chian Hsiao, Taipei City (TW); Yanping Zhou, New Taipei City (TW)

(21) Appl. No.: 19/215,354

(22) Filed: May 22, 2025

Related U.S. Application Data
(60) Provisional application No. 63/650,948, filed on May 23, 2024.

(51) Int. Cl. C12M 1/42; C12M 1/12; C12M 3/06

(52) U.S. Cl. CPC C12M 35/02; C12M 23/16; C12M 25/06

(57) ABSTRACT

A cell culture structure and a cell culture device are provided. The cell culture structure includes a substrate having a top surface and a bottom surface. A plurality of micro recesses are recessed from the top surface toward the bottom surface of the substrate, in which a recess width of each of the micro recesses decreases from the top surface toward the bottom surface to form an inclined sidewall. The inclined sidewall of each of the micro recesses is formed with a nano structure conforming to and covering a three-dimensional contour of the corresponding micro recess. Each of the micro recesses and the corresponding nano structure collectively form a three-dimensional culture space.

### CROSS-REFERENCE TO RELATED PATENT APPLICATION

This application claims the benefit of priority to the U.S. Provisional Patent Application Ser. No. 63/650,948, filed on May 23, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a cell culture structure, and more particularly to a cell culture structure and a cell culture device.

### BACKGROUND OF THE DISCLOSURE

In the related art, cell culture is mostly carried out in two-dimensional plane culture, in which cells are cultured on a common culture dish or a plane chip. However, this method can easily lead to differences in cell proliferation, differentiation and functional performance compared to physiological conditions. In addition, cells tend to stack in two-dimensional culture, resulting in uneven transfer of nutrients and metabolic waste, thereby increasing cell mortality. Although some studies have attempted to use micro-structured substrates to improve cell attachment and transfection efficiency, there are still deficiencies in terms of single cell separation and cell recovery convenience.

Accordingly, there is a need to develop a technology being capable of uniform single cell partitioning for the application of cell culture. Further, it is anticipated that advanced biotechnological functions, such as delivering genetic materials to individual cells while ensuring high cell viability, could be achieved.

### SUMMARY OF THE DISCLOSURE

In response to the above-referenced technical inadequacies, the present disclosure provides a cell culture structure and a cell culture device for culturing suspension cells and adhesion cells.

In order to solve the above-mentioned problems, one of the technical aspects adopted by the present disclosure is to provide a cell culture structure.

The cell culture structure includes a substrate having a top surface and a bottom surface opposite to each other. The cell culture structure further has a plurality of micro recesses being recessed from the top surface toward the bottom surface of the substrate, in which a recess width of each of the micro recesses decreases from the top surface toward the bottom surface to form an inclined sidewall.

The inclined sidewall of each of the micro recesses is formed with a nano structure conforming to and covering a three-dimensional contour of the corresponding micro recess. Each of the micro recesses and the corresponding nano structure collectively form a three-dimensional culture space.

Preferably, each of the micro recesses is an inverted conical recess or an inverted hemispherical recess.

Preferably, each of the micro recesses has an opening width ranging from 3 micrometers to 30 micrometers, and a recess depth ranging from 1 micrometer to 30 micrometers.

Preferably, the nano structure includes a plurality of nano pillars formed on the inclined sidewall, and each of the nano pillars has a pillar height not greater than 5 micrometers.

Preferably, the plurality of nano pillars in each of the micro recesses extend from a surface of the inclined sidewall along a local surface normal direction, in which an arrangement of the nano pillars conforms to and fully covers the stereo (3D) contour of the micro recess so as to form a nano-scale pillar structure layer that surrounds toward an inner side of the micro recess.

Preferably, a pillar width (or pillar diameter) of each of the nano pillars ranges from 10 nanometers to 500 nanometers, and a distance between any two adjacent nano pillars ranges from 50 nanometers to 200 nanometers.

Preferably, the substrate is a single-crystal silicon substrate having an (1,0,0) silicon crystal orientation, and a first angle between each of the nano pillars and the surface of the inclined sidewall ranges from 80 degrees to 100 degrees.

Preferably, each of the micro recesses further has a biocompatible polymer film covered on the nano structure, in which the biocompatible polymer film is covalently bonded to the nano structure, and a thickness of the biocompatible polymer film ranges from 5 nanometers to 50 nanometers.

Preferably, a bottom of each of the micro recesses forms a through-hole that penetrates the bottom surface of the substrate, and a hole diameter of the through-hole ranges from 1 micrometer to 5 micrometers.

Preferably, the plurality of micro recesses are arranged in a matrix or staggered pattern, and a distance between any two adjacent micro recesses ranges from 10 micrometers to 65 micrometers.

Preferably, the cell culture structure further includes a hydrophilic surface (e.g., a layer of poly(ethylene glycol) (PEG), a plasma treatment surface or a layer of silicon nitride deposition) formed on the top surface of the substrate in the area between the plurality of micro recesses.

The embodiment of the present disclosure further discloses a cell culture device that includes an upper cover plate, and the cell culture structure as described above. An opening of each of the micro recesses faces toward the upper cover plate. A flow channel space is formed between the upper cover plate and the cell culture structure to introduce a cell suspension or culture fluid into the plurality of micro recesses of the substrate or an exit for waste.

Preferably, the upper cover plate is made of a transparent material.

Preferably, the upper cover plate is provided with an upper electrode, and the cell culture device is provided with a lower electrode; and when the micro recesses carry cells, the upper electrode and the lower electrode are used to apply an electric field to the cells.

Therefore, through the afore-mentioned structural design, the cell culture structure provided by the present disclosure can effectively partition single cells (up to five cells) in each micro recess, and the three-dimensional culture space of the micro-nano hybrid structure is conducive to cell cultivation and improves the survival rate of the cells. Furthermore, in the application of cell puncture, the device can use the recesses to concentrate the electric field and the nano structures to enhance cell membrane permeability, reducing the voltage required by conventional planar bulk electroporation techniques and effectively improving the delivery efficiency of gene materials into single cells, as well as cell viability.

Moreover, the addition of a bottom through-hole and a biocompatible polymer modification layer further endows the substrate with greater flexibility and multifunctional potential.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a cell culture structure according to an embodiment of the present disclosure;

FIG. 2 is a partially enlarged view of a single three-dimensional culture space (i.e., a single cell culture well) in FIG. 1;

FIG. 3A is a cross-sectional view of the cell culture structure according to the embodiment of the present disclosure;

FIG. 3B is a schematic view of a variation of the a cell culture structure having a through-hole according to the embodiment of the present disclosure;

FIG. 4 is a top view of the cell culture structure according to the embodiment of the present disclosure;

FIG. 5A is a partially enlarged view of region V in FIG. 3A;

FIG. 5B is a schematic view of another variation of the embodiment of the present disclosure, in which the nano structure is covered by a biocompatible polymer film;

FIG. 6A is an SEM photograph at 3,000× magnification of a cross-sectional view of an inverted conical recess of the substrate according to the embodiment of the present disclosure;

FIG. 6B is an SEM photograph at 3,000× magnification showing the inverted conical recess of the substrate in a top view according to the embodiment of the present disclosure;

FIG. 6C is a partially enlarged SEM photograph at 30,000× magnification of the nano pillars on the inclined sidewall of the inverted conical recess of the substrate according to the embodiment of the present disclosure;

FIG. 6D is an SEM photograph of a transition region between an apex corner area of the inverted conical recess and the bottom of the recess;

FIG. 6E is an SEM photograph at 30,000× magnification of the nano pillars on the inclined sidewall of the inverted conical recess;

FIG. 7A is an SEM photograph showing an array of inverted pyramid recesses on the substrate with cells loaded according to the embodiment of the present disclosure;

FIG. 7B is an SEM photograph showing a cell-loaded state inside a single inverted pyramid recess;

FIG. 8 is a schematic view of a cell culture device according to the embodiment of the present disclosure; and

FIG. 9 is a schematic view illustrating the accommodation of a single cell by the cell culture device according to the embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### [Cell Culture Structure]

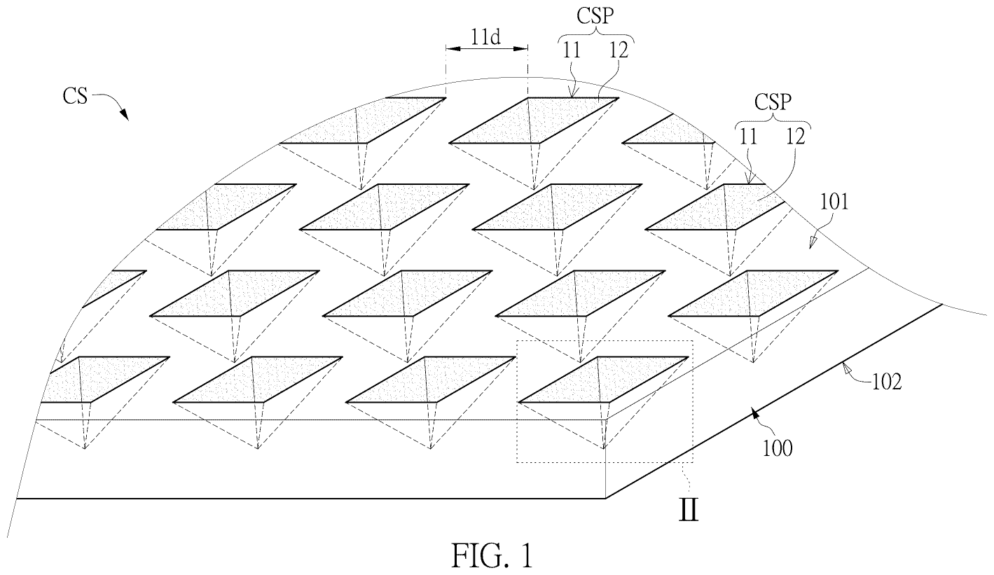

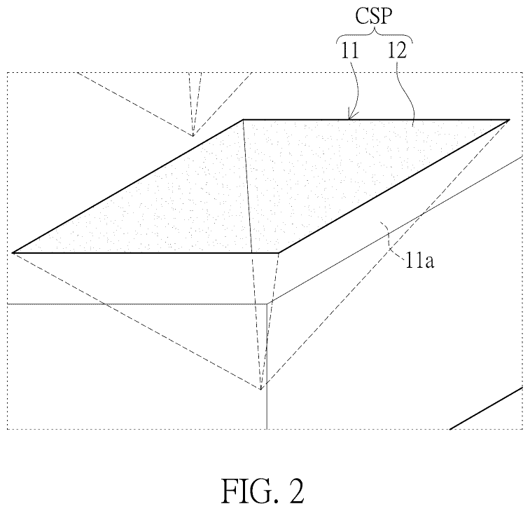

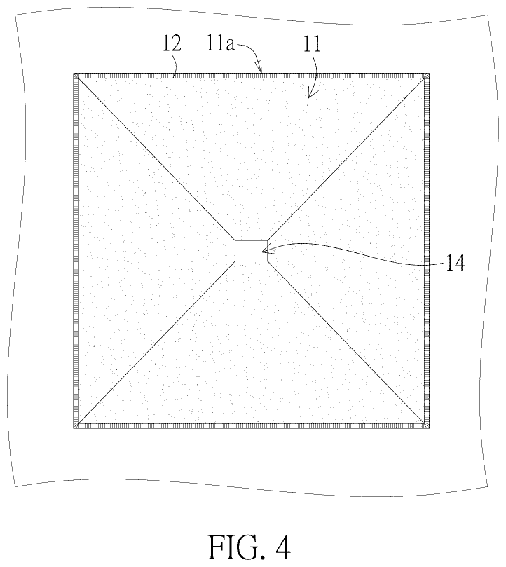

Referring to FIGS. 1 to 7, the first embodiment of the present disclosure provides a cell culture structure CS for cell culture, which is primarily used to achieve single cell partition and culture. Through the structural design of the substrate, the substrate can effectively separate cells, and increase the uniformity and efficiency of gene delivery while reducing the required voltage and minimizing cell damage, thereby addressing issues such as inadequate delivery efficiency, uneven electric fields, and poor cell viability in the relevant art.

More specifically, the cell culture structure CS includes a substrate 100. The substrate 100 is a conductive or conductively treated substrate.

The cell culture structure CS includes a plurality of micro recesses 11. The plurality of micro recesses 11 are recessed from a top surface 101 toward a bottom surface 102 of the substrate 100, and are arranged in a matrix or staggered pattern with spacing. Each of the micro recesses 11 has a recess width that decreases from the top surface 101 toward the bottom surface 102 of the substrate 100, thereby forming an inclined sidewall 11a.

Each of the micro recesses 11 is an inverted conical recess or an inverted hemispherical recess. The inverted conical recess can be an inverted pyramidal (quadrangular) conical recess, an inverted triangular conical recess, or an inverted circular conical recess. In the present embodiment, each of the micro recesses 11 is an inverted pyramidal recess.

Furthermore, the inclined sidewall 11a of each of the micro recesses 11 is formed with a nano structure 12 that conforms to and covers a three-dimensional (3D) contour of the corresponding micro recess 11.

Each of the micro recesses 11 and the corresponding nano structure 12 on the inclined sidewall 11a together define a three-dimensional culture space CSP.

The three-dimensional culture space CSP can accommodate at least one cell (e.g., one to five cells), which is beneficial for single cell loading and culture (e.g., nanostructures 12 can improve cell adhesion and interactions).

In some embodiments of the present disclosure, the material of the substrate 100 is preferably single-crystal silicon, more preferably single-crystal silicon having an (1,0,0) silicon crystal lattice orientation, to facilitate processes such as photolithography and etching process.

However, the material of the substrate 100 can also be selected from glass, quartz, sapphire, or other base materials, according to usage requirements. If a high-impedance material is adopted, a conductive film can be formed on a back side of the substrate, for instance a titanium-gold (Ti/Au) alloy film or a gold (Au) film, to ensure conductivity of the substrate, but the present disclosure is not limited thereto.

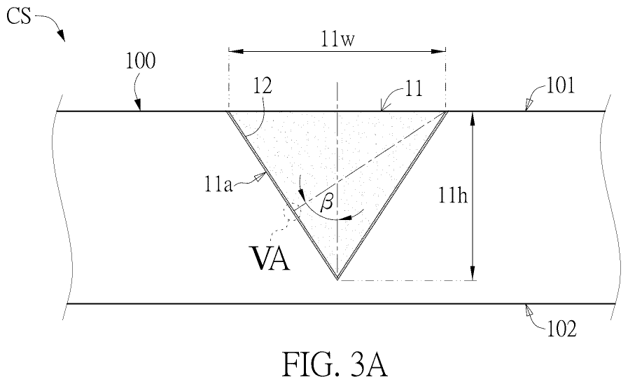

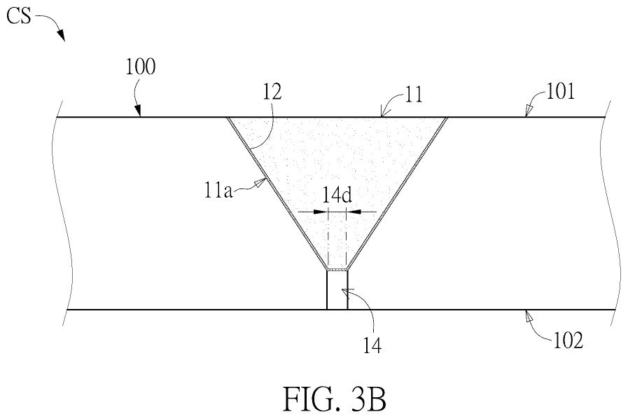

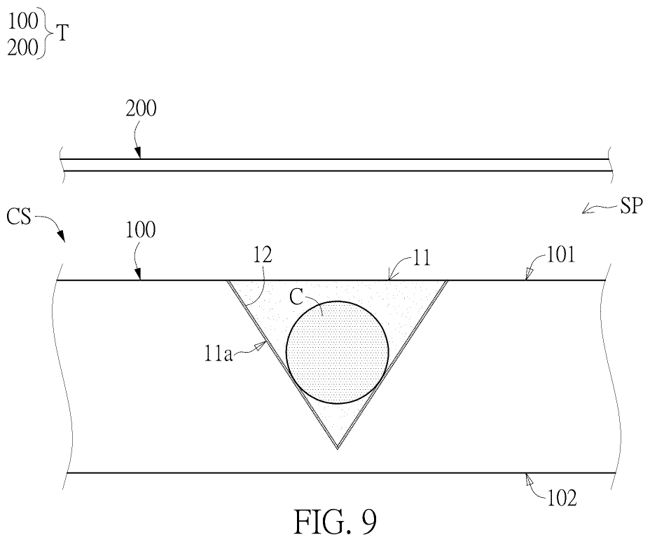

Referring to FIG. 3A, in some embodiments of the present disclosure, each of the micro recesses 11 (e.g., the inverted pyramidal recess) has an opening width 11w ranging from 3 micrometers to 30 micrometers, and preferably ranging from 20 micrometers to 25 micrometers. Each of the micro recesses 11 has a recess depth 11h ranging from 1 micrometer to 30 micrometers, and preferably ranging from 20 micrometers to 30 micrometers, such that each of the micro recesses 11 can accommodate 1 to 5 cells, and more preferably 1 to 2 cells (as shown in FIG. 9). Moreover, the plurality of micro recesses 11 can be arranged with spacing such that a recess pitch 11d between each micro recess 11 and an adjacent micro recess 11 is about 10 micrometers to 65 micrometers, which can effectively distribute and isolate cells.

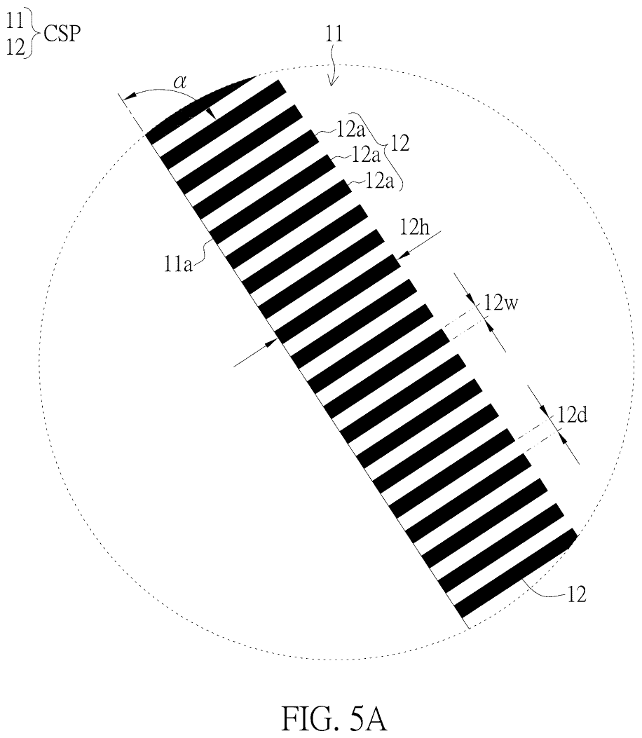

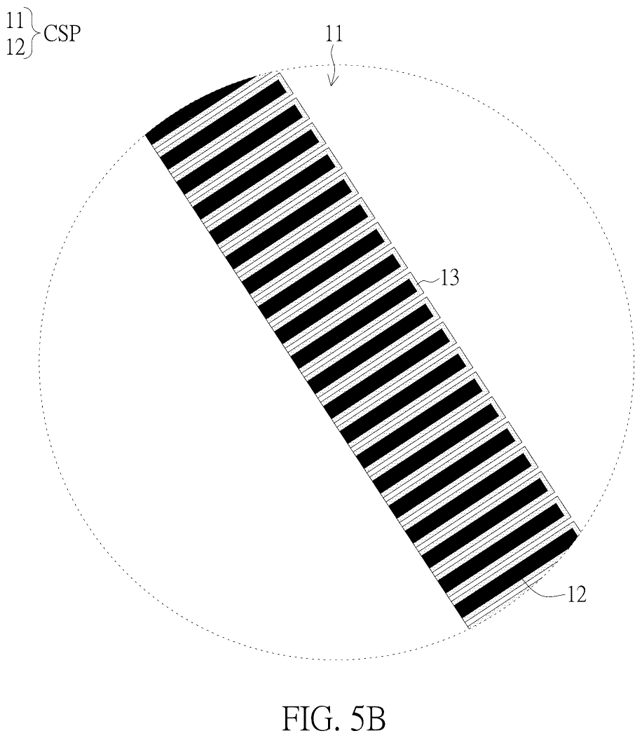

Referring to FIG. 5A, furthermore, each of the micro recesses 11 (e.g., the inverted conical recess) is provided with the nano structure 12 on the inclined sidewall 11a. The nano structure 12 includes a plurality of nano pillars 12a that stand upright on and densely populate the surface of the inclined sidewall 11a. For example, the nano pillars 12a can be formed as an array of nano pillars (e.g., an array of multiple silicon nano pillars) or an array of nano wires (e.g., an array of multiple silicon nano wires) by dry etching or wet etching on the inclined sidewall 11a of the micro recess 11. The nano pillars 12a feature a high aspect ratio, forming a brush-like surface.

In other embodiments of the present disclosure, the plurality of nano pillars 12a of the nano structure 12 can also be carbon nanotube arrays or arrays of metallic pillar structures (that can be used as a three-dimensional electrode) formed by a vapor deposition process.

For example, in some embodiments of the present disclosure, the nano pillars 12a can be fabricated by metal-assisted chemical etching (MACE), deep reactive ion etching (DRIE), or by selectively depositing a metal thin film followed by etching. However, the present disclosure is not limited thereto. Alternatively, in a variant embodiment of the present disclosure, the nano structure 12 on the inclined sidewall 11a of each micro recess 11 can be nano holes or nano islands.

Furthermore, each nano pillar 12a of the nano structure 12 has a pillar height 12h not greater than 5 micrometers, preferably not greater than 3 micrometers, and more preferably ranging from 1.0 micrometer to 3 micrometers, to avoid excessive cell penetration or difficulty in cell retrieval.

In addition, a pillar width 12w of each nano pillar 12a ranges from 10 nanometers to 500 nanometers, preferably from 10 nanometers to 200 nanometers, and more preferably from 30 nanometers to 65 nanometers.

The distance 12d between any two adjacent nano pillars 12a ranges from 50 nanometers to 200 nanometers, and preferably from 50 nanometers to 150 nanometers, so as to provide a dense and uniform set of contact points for cell adhesion, thereby enhancing membrane permeability of cells. If the distance is too large, cells may adhere, making them difficult to remove or causing death.

In some embodiments of the present disclosure, the growth direction of the plurality of nano pillars 12a formed on the inclined sidewall 11a of each micro recess 11 (e.g., the inverted conical recess) is affected by the crystal lattice plane of the substrate 100, exhibiting specific directionality.

For example, the substrate 100 is preferably made of single-crystal silicon, and more preferably single-crystal silicon having an (1,0,0) silicon crystal lattice orientation.

Specifically, the plurality of nano pillars 12a are arranged standing upright from the surface of the inclined sidewall 11a of each micro recess 11 (e.g., an inverted conical recess). A first angle α between each nano pillar 12a and the surface of the inclined sidewall 11a is from 80 degrees to 100 degrees, and preferably from 85 degrees to 95 degrees.

From another viewpoint, a second angle β is formed between a virtual extension line of each nano pillar 12a and a normal direction of the substrate 100, and the second angle β ranges from 35 degrees to 55 degrees, preferably from 40 degrees to 50 degrees, but the present disclosure is not limited thereto.

In other words, the growth direction of the plurality of nano pillars 12a is influenced by the crystal lattice orientation of the substrate 100, exhibiting a special growth angle that is perpendicular or nearly perpendicular to the inclined surface 11a. Through the specific growth direction of the nano pillars, the contact area and contact points between the cells and the nano structures can be effectively increased, and the efficacy of single cell culture is improved. However, the present disclosure is not limited to the specific growth direction of the nano pillars described herein, any directionally controlled nano pillar structure derived from crystal orientation adjustments that achieves a similar effect is also encompassed within the scope of the present disclosure.

From another angle, the plurality of nano pillars 12a extend from the surface of the inclined sidewall 11a of each micro recess 11 in the local surface normal direction and are arranged to conform to and completely cover the three-dimensional (3D) contour of the micro recess 11, thereby forming a nano pillar structure layer that surrounds the inner side in three dimensions within the recess. Accordingly, whether the micro recess 11 is an inverted conical, inverted pyramidal, inverted triangular, or inverted circular conical shape, the nano pillars 12a naturally form a three-dimensional inwardly convergent structure along the contour of the sidewall. This effectively increases multi-directional contact between the nano pillars 12a, which is beneficial for cell adhesion and culture.

Referring to FIG. 5B, in one embodiment of the present disclosure, in order to prevent mechanical damage to cells caused by the nano structure 12 and simultaneously provide a functional surface capable of adsorbing and immobilizing nucleic acids or proteins, a biocompatible polymer film 13 can be formed on the nano structure 12 and surrounds each of the nano pillars 12a. The thickness of the polymer film 13 ranges from 5 nanometers to 50 nanometers, preferably from 10 nanometers to 20 nanometers. The polymer film 13 is formed on the nano structure 12 by covalent bonding.

For example, the material of the biocompatible polymer film 13 can include silane-derived polymer such as silane conjugated poly(ethylene glycol) (silane-PEG) or amino-silane. After surface activation by solution such as piranha or surface plasma treatment, the polymer film 13 is capable of covalently bonding or physically adsorbing to the nanostructures, where various negatively charged biomolecules, such as various charged molecules, negatively charged oligo nucleotide or deoxyribonucleic acid (DNA), thereby further enhancing the retention of biomolecules (particles) before the cell culture, but the present disclosure is not limited thereto.

Referring to FIG. 3B, in another embodiment of the present disclosure, the bottom of each micro recess 11 (e.g., an inverted conical recess) further forms a through-hole 14 that penetrates the bottom surface 102 of the substrate 100. The hole diameter 14d of the through-hole 14 ranges from 1 micrometer to 5 micrometers, and preferably from 2 micrometers to 3 micrometers.

By means of the through-hole 14, fluidic channels in a microfluidic device disposed below the substrate can be connected so that an operator can supply or remove culture medium, reagents, or other necessary solutions in real time from below the substrate during gene delivery. This achieves real-time and precise microfluidic control of the cell culture environment. However, in other embodiments of the present disclosure, if no real-time fluid exchange is required, the through-hole may be omitted.

In an embodiment of the present disclosure, a hydrophilic surface (e.g., a layer of poly(ethylene glycol) (PEG), a plasma treatment surface or a layer of silicon nitride deposition) is formed on the top surface 102 of the substrate 100 in the area between the plurality of micro recesses 11, which can help prevent cells from non-specific binding on the top surface of the substrate 100.

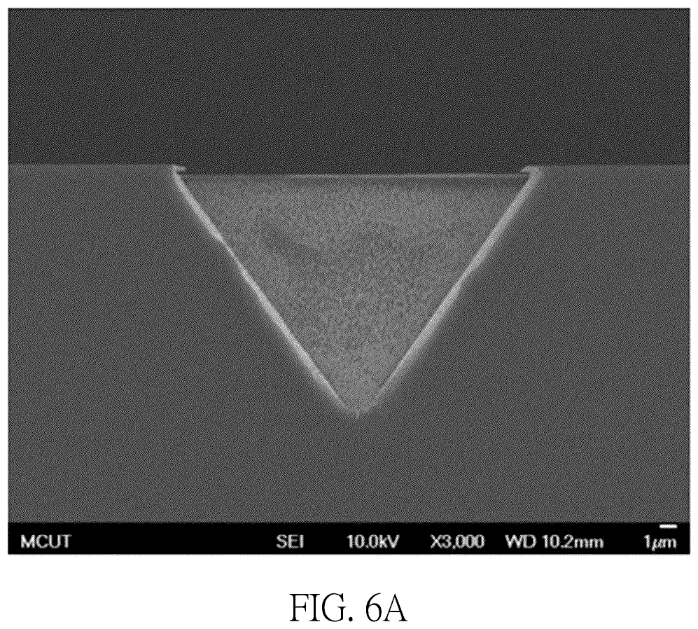

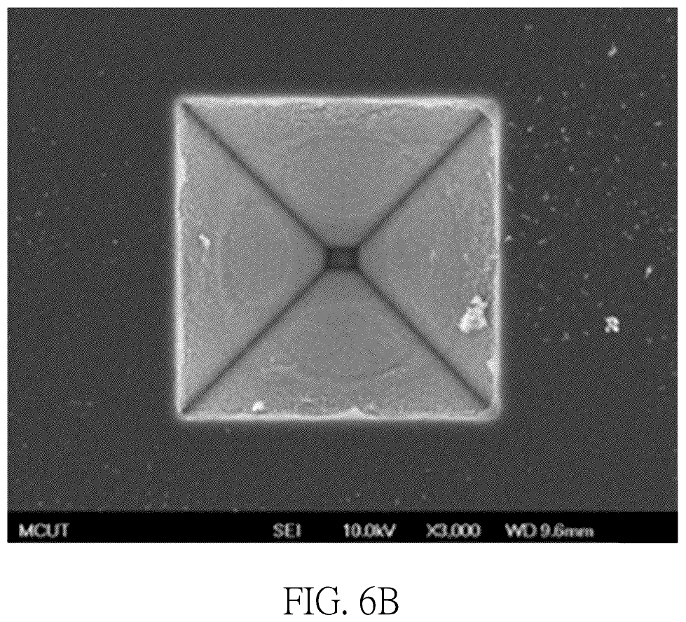

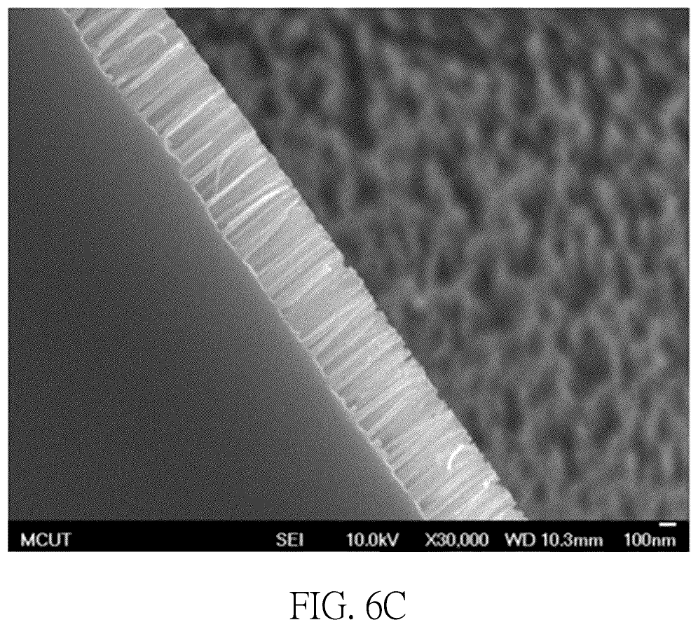

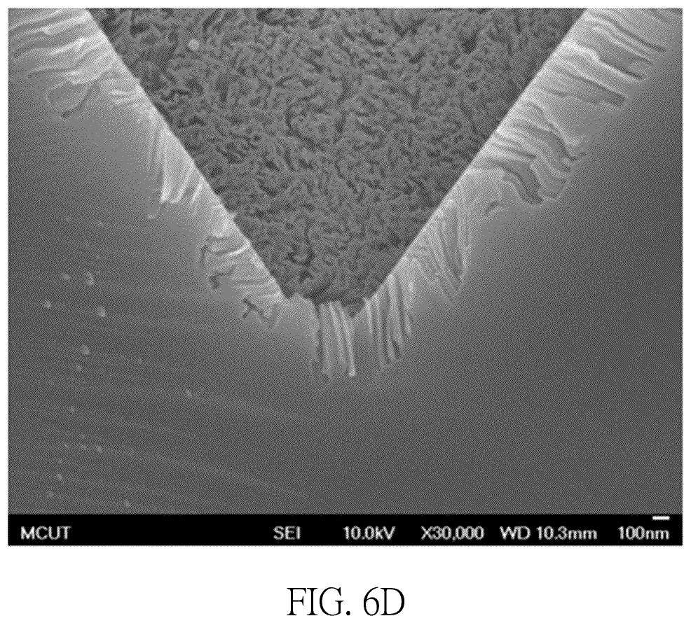

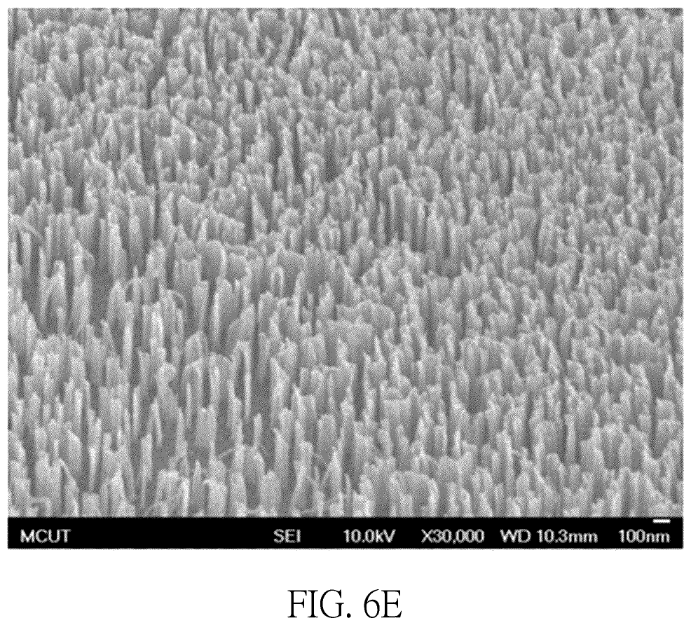

Referring to FIGS. 6A to 6E, these are relevant scanning electron microscope (SEM) images of the substrate according to an embodiment of the present disclosure. FIG. 6A shows a cross-sectional SEM image of an inverted conical recess of the substrate according to the embodiment of the present disclosure at 3,000× magnification. It can be observed that the inclined sidewall of the recess has multiple nano pillars with a brush-like appearance.

FIG. 6B shows a top-view SEM image at 3,000× magnification of the inverted conical recess of the substrate according to the embodiment of the present disclosure. Four edges are symmetrically aligned, and the apex converges at the center, thereby providing an environment conducive to positioning single cells.

FIG. 6C is a partially enlarged SEM image of the nano pillars on the inclined sidewall of the inverted conical recess at 30,000× magnification, revealing a uniformly arranged high-aspect-ratio nano pillar array that extends perpendicularly from the inclined sidewall surface, thereby increasing the cell attachment area.

FIG. 6D is an SEM image at 30,000× magnification showing the nano pillars in the corner region of the apex of the inverted conical recess transitioning to the bottom of the recess. The nano pillars continuously grow along the edge of the inclined sidewall without significant detachment at the ends, demonstrating that the nano structure and the micro recess are integrally formed to provide a stable culture space.

FIG. 6E is a top-view SEM image at 30,000× magnification of the nano pillars on the inclined sidewall of the inverted conical recess, showing a densely packed brush-like distribution that provides an efficient surface for carrier attachment while reducing mechanical damage during cell retrieval.

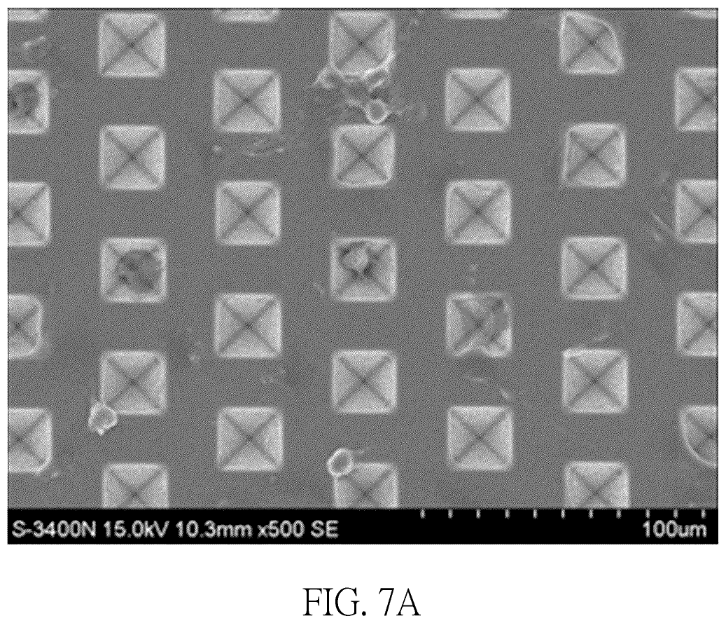

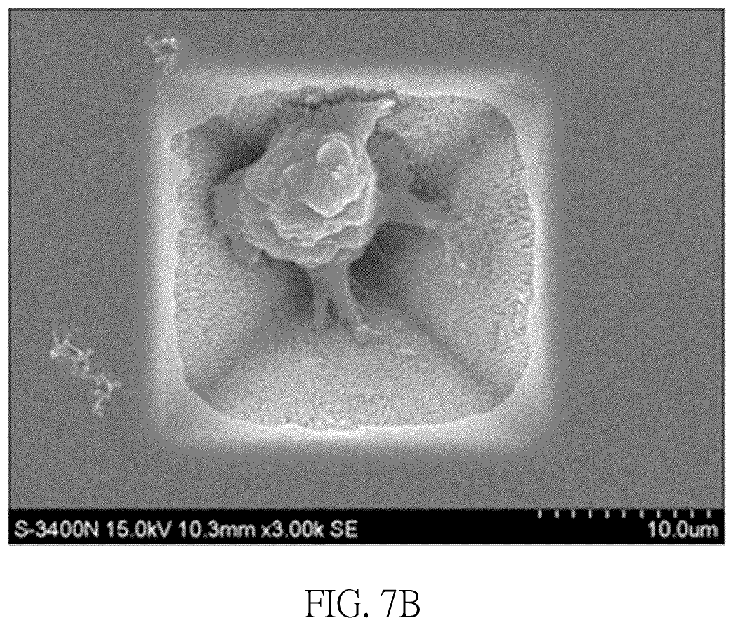

Referring to FIGS. 7A and 7B, these are SEM images showing cells loaded onto the substrate according to the embodiment of the present disclosure. FIG. 7A is an SEM image at 500× magnification of an array of inverted conical recesses on the substrate of the embodiment of the present disclosure with cells loaded. It can be observed that most recesses successfully contain a single cell, indicating that the structure effectively guides cells to settle while preventing stacking, thereby achieving single-cell distribution.

FIG. 7B is an SEM image at 3,000× magnification showing the state of cell adhesion inside a single inverted conical recess. The cell adheres to the bottom of the recess and exhibits apparent pseudopodia extending toward the surface of the nano structure, confirming favorable interaction between the cell and the inner surface of the recess. This assists in enhancing the stability of cell culture.

In general, the cell culture structure CS in the embodiment of the present disclosure effectively partitions single cells (up to five) through the above-mentioned multi-level structural design, and the three-dimensional culture space of the micro-nano hybrid structure is conducive to improve the viability of single cell culture.

It is worth noting that animal cells exhibit distinct biological responses and behaviors at different dimensional scales. Therefore, animal cells have commonly been utilized in biotechnology as products or research tools across various fields. For instance, microwell arrays constitute important tools for high-throughput analysis of living single cells. By confining cells within specific micron-scale arrays, spatial and temporal information regarding cellular responses can be obtained, and even single-cell genetic characteristics can be purified within these microwell arrays for medical analyses. At the nanoscale, researchers have identified different cell functionalities, such as controlling cell adhesion, influencing the absorption of substances onto cell surfaces, and altering cell migration rates. However, among these previous studies and inventions, only a limited number of researchers have specifically analyzed the interaction between living single cells confined in micron-scale recesses and corresponding nanoscale structures. Although nanoscale structures are known to regulate living cell adhesion, deformation, and signal transduction, the integration of nanoscale structures into high-throughput platforms has remained challenging. Therefore, a significant gap still exists in systematic single-cell behavior studies, such as long-term single-cell culture, proliferation research, and differentiation monitoring.

The embodiment of the present disclosure provides a hybrid structural feature at micro- and nano-scales. The hybrid structural feature employs micron-scale recesses to fulfill the requirements of accommodating single cells, while nanoscale structures formed within the micron-scale recesses facilitate interactions with the single cells. The hybrid micro- and nano-scale structural feature of the embodiment of the present disclosure has been successfully realized on a single-crystal silicon substrate. Under cell culture conditions utilizing the hybrid structural feature, single-cell survival rates as high as 90% have been achieved. The resulting high cell survival rate demonstrates that the hybrid micro- and nano-scale structural feature maintains biocompatibility and exhibits potential for commercial applications in future high-throughput cell culture and analysis technologies.

However, the structural details described in the embodiment of the present disclosure are provided for illustrative purposes and are not intended to limit the scope of the present disclosure. Any equivalent modifications and alterations not departing from the technical concepts of the present disclosure should be included within the scope of the present disclosure.

### [Cell Culture Device]

A second embodiment of the present disclosure provides a cell culture device T, which integrates the cell culture structure CS that includes the substrate 100 and the three-dimensional culture space E as described in the embodiment of the present disclosure above. The platform is designed to utilize the unique structure and function of the substrate 100 to achieve high-efficiency and uniform single-cell-scale loading, culture, gene transfection and biomolecule introduction, specifically applicable to cell partition and cell membrane perforation.

More specifically, the cell culture device T includes the aforementioned cell culture structure CS as the core component for cell partition.

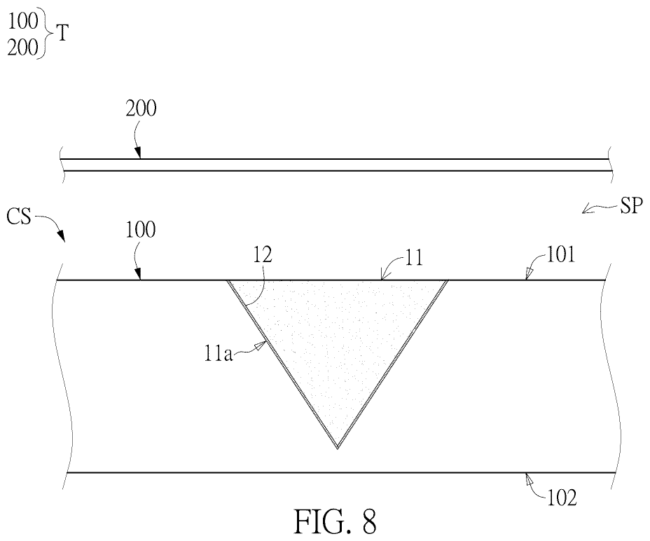

Referring to FIGS. 8 and 9, the cell culture device T includes an upper cover plate 200 and the cell culture structure CS (which includes the substrate 100 and the three-dimensional culture spaces E). An opening of each of the micro recesses 11 faces toward the upper cover plate 200. A flow channel space SP is formed between the upper cover plate 200 and the substrate 100 of the cell culture structure CS to introduce a cell suspension or culture fluid into the plurality of micro recesses 11 of the substrate 100.

In some embodiments of the present disclosure, the upper cover plate 200 can optionally be made of a material with both transparency and conductivity, such as indium tin oxide (ITO) glass, so as to facilitate real-time optical monitoring and image analysis during gene delivery. The upper cover plate 200 can be provided with an upper electrode (not shown). The cell culture structure CS with the three-dimensional culture space CSP can be provided with a lower electrode that forms an effective electric field circuit through the metallization of the nanostructures 12 or the metallization or doping to the nanostructures such as silicon nanowires. When an electric voltage is applied between the upper and lower electrodes, a local and uniform electric field is generated in each of the micro recesses 11 (e.g., the inverted conical recess), providing precise, low damage electroporation to single cells.

Furthermore, the cell culture device T is used for delivering or discharging aqueous solution such as cell culture media or cell suspension. The cell culture device T can include one or more micro-channels and corresponding inlet/outlet ports. Through fluid dynamics control in the micro-channels, cells C are uniformly introduced into the plurality of micro recesses 11 of the cell culture structure CS (as shown in FIG. 9), achieving accurate single-cell-level distribution.

The size and design of the microchannels can be adjusted as needed. Preferably, the width of the microchannels is 50 micrometers to 500 micrometers, more preferably 100 micrometers to 200 micrometers, in order to precisely control the flow rate and distribution density of cells, but the present disclosure is not limited thereto. In addition, the microfluidic system can also be connected to the through-holes 14 at the bottom of the substrate 100 (see FIG. 3B) to further achieve bidirectional fluid exchange. The microfluidic system can provide real-time replacement of culture media or reagents and removal of waste solutions on demand.

In some embodiments, an electrode control module can include an adjustable power supply and an electric field waveform generator, which control the magnitude, waveform, frequency, and duration of the voltage applied to the upper and lower electrodes in order to achieve electroporation.

In some embodiments, the cell culture device T may additionally be equipped with an optical detection module, such as a fluorescence microscopy system, to observe and analyze cell status in real time.

In some embodiments of the present disclosure, the micro recesses 11 of the cell culture structure CS in the cell culture device T can be used for culturing cells, in which the survival rate of cells cultured for one to three days ranges from 20% to 99%.

The cells accommodated therein can be adherent cells or suspension cells. The accommodated cells can be released from the device by trypsin or EDTA digestion followed by centrifugation. Subsequently, the cell viability of cells released from the cavity by centrifugation ranges between 50% and 90%.

Overall, by integrating the cell culture structure CS, the microfluidic system, the electrode control module, and the optical detection module, the cell culture device T of the present disclosure provides a highly controllable, high-throughput gene delivery solution at the single-cell level. The solution is applicable to various research and application fields requiring precise regulation of gene expression, including cell therapy, gene editing, and drug screening, among others, but the scope of the present disclosure is not limited thereto. Any variations or modifications made based on the same structural concepts or module combinations to achieve similar effects are intended to fall within the scope of the protection sought by the present disclosure.

### Beneficial Effects of the Embodiment

In conclusion, through the aforementioned structural arrangement, the cell culture structure of the present disclosure offers the following technical effects: (1) The cell culture structure can effectively accommodate single cell (up to five cells) in each micro recess, and the three-dimensional culture space of the micro-nano hybrid structure is conducive to cell cultivation and improves the survival rate of the cells. (2) The nano structure on the sidewall of the recess forms a three-dimensional coating of high-aspect-ratio nano pillars, which increase the contact area of the cell membrane and provide multiple directions of electric field interaction points. This configuration can improve the flux and uniformity of gene or biomolecule entry into the cell. (3) Each recess and its nano structure constitute an independent three-dimensional culture space, enabling reproducible and quantitative gene delivery at the single-cell scale, thereby effectively improving the reproducibility of batch experiments.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A cell culture structure, comprising:
a substrate having a top surface and a bottom surface; and
a plurality of micro recesses being respectively recessed from the top surface toward the bottom surface of the substrate, wherein a recess width of each of the micro recesses decreases from the top surface toward the bottom surface to form an inclined sidewall;
wherein the inclined sidewall of each of the micro recesses is formed with a nano structure conforming to and covering a three-dimensional contour of the corresponding micro recess, and wherein each of the micro recesses and the corresponding nano structure collectively form a three-dimensional culture space.

2. The cell culture structure according to claim 1, wherein each of the micro recesses is an inverted conical recess or an inverted hemispherical recess.

3. The cell culture structure according to claim 1, wherein each of the micro recesses has an opening width ranging from 3 micrometers to 30 micrometers, and a recess depth ranging from 1 micrometer to 30 micrometers.

4. The cell culture structure according to claim 1, wherein the nano structure includes a plurality of nano pillars formed on the inclined sidewall, and each of the nano pillars has a pillar height not greater than 5 micrometers.

5. The cell culture structure according to claim 4, wherein the plurality of nano pillars in each of the micro recesses extend from a surface of the inclined sidewall along a local surface normal direction, and wherein an arrangement of the nano pillars conforms to and fully covers the stereo contour of the micro recess so as to form a nano-scale pillar structure layer that surrounds toward the inner side of the micro recess.

6. The cell culture structure according to claim 4, wherein a pillar width of each of the nano pillars ranges from 10 nanometers to 500 nanometers, and a distance between any two adjacent nano pillars ranges from 50 nanometers to 200 nanometers.

7. The cell culture structure according to claim 4, wherein the substrate is a single-crystal silicon substrate having an (1,0,0) silicon crystal orientation, and a first angle between each of the nano pillars and the surface of the inclined sidewall ranges from 80 degrees to 100 degrees.

8. The cell culture structure according to claim 1, wherein each of the micro recesses further has a biocompatible polymer film covered on the nano structure, and wherein the biocompatible polymer film is covalently bonded to the nano structure, and a thickness of the biocompatible polymer film ranges from 5 nanometers to 50 nanometers.

9. The cell culture structure according to claim 1, wherein a bottom of each of the micro recesses forms a through-hole that penetrates the bottom surface of the substrate, and a hole diameter of the through-hole ranges from 1 micrometer to 5 micrometers.

10. The cell culture structure according to claim 1, wherein the plurality of micro recesses are arranged in a matrix or staggered pattern, and a distance between any two adjacent micro recesses ranges from 10 micrometers to 65 micrometers.

11. The cell culture structure according to claim 1, further comprising: a hydrophilic surface formed on the top surface of the substrate in the area between the plurality of micro recesses.

12. A cell culture device, comprising:
an upper cover plate;
a cell culture structure disposed at an interval below the upper cover plate; wherein the cell culture structure includes:
a substrate having a top surface and a bottom surface; and
a plurality of micro recesses being respectively recessed from the top surface toward the bottom surface of the substrate, wherein a recess width of each of the micro recesses decreases from the top surface toward the bottom surface to form an inclined sidewall, wherein the inclined sidewall of each of the micro recesses is formed with a nano structure conforming to and covering a three-dimensional contour of the corresponding micro recess, and wherein each of the micro recesses and the corresponding nano structure collectively form a three-dimensional culture space, and wherein an opening of each of the micro recesses faces toward the upper cover plate;
wherein a flow channel space is formed between the upper cover plate and the cell culture structure to introduce a cell suspension or culture fluid into the plurality of micro recesses of the substrate or as an exit for waste.

13. The cell culture device according to claim 12, wherein the upper cover plate is made of a transparent material.

14. The cell culture device according to claim 12, wherein the upper cover plate is provided with an upper electrode, and the cell culture device is provided with a lower electrode; and when the micro recesses carry cells, the upper electrode and the lower electrode are used to apply an electric field to the cells.
